(19) United States
(12) Patent Application Publication
(10) Pub. No.: US 2026/0363725 A1
(43) Pub. Date: Nov. 19, 2026

(54) DEVICE AND PERCUTANEOUS METHODS FOR CLEARING EPIDURAL ADHESIONS

(71) Applicant: Rhode Island Hospital, Providence, RI (US)

(72) Inventor: Albert TELFEIAN, Providence, RI (US)

(21) Appl. No.: 19/133,693

(22) PCT Filed: Nov. 28, 2023
(86) PCT No.: PCT/US2023/081371

§ 371 Date: May 28, 2025

Related U.S. Application Data
(60) Provisional application No. 63/385,358, filed on Nov. 29, 2022.

(51) Int. Cl. A61B 17/3207; A61B 17/32

(52) U.S. Cl. CPC A61B 17/320725; A61B 17/320016

(57) ABSTRACT

Devices and methods for clearing adhesions in a patient's epidural space. A surgeon uses the devices to clear adhesions and may then place electrodes within the patient's epidural space. The devices help surgeons to overcome the challenge of deploying a percutaneous stimulator or recording lead that takes on a larger final conformation once it is in the final epidural location. The devices include an inner lead member, an outer tube configured to receive the inner lead member therein and an expandable member operably connected to a distal end of the inner lead member and moveably positioned within the outer tube. The devices are moveable between an undeployed position in which the expandable member is at least partially contained within the outer tube, and a deployed position in which the expandable member is expanded to extend outside of the outer tube and configured to clear adhesions within the patient's epidural space.

### RELATED APPLICATION

This application claims the benefit of priority to U.S. Provisional Patent Application No. 63/385,358, filed Nov. 29, 2022, the disclosure of which is incorporated by reference herein in its entirety.

### FIELD OF THE INVENTION

The invention relates to percutaneous methods for clearing epidural adhesions to clear the epidural space prior to the implantation of an electrode, and devices for performing such methods.

### BACKGROUND OF THE INVENTION

Neuromodulation has tremendous future applications in medicine. Electrodes can be placed in the epidural space in a patient's brain and/or spine. Currently, one challenge in deploying epidural electrodes in the brain or spine, in a minimally invasive way, is to get them to expand to cover the epidural space because they can be restricted by epidural adhesion.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

According to an exemplary embodiment of the innovation, a device for clearing adhesions in a patient's epidural space includes an inner lead member; an outer tube configured to receive the inner lead member therein; and an expandable member operably connected to a distal end of the inner lead member and moveably positioned within the outer tube. The device is moveable between an undeployed position in which the expandable member is at least partially contained within the outer tube, and a deployed position in which the expandable member is expanded to extend outside of the outer tube and configured to clear adhesions within the patient's epidural space. In an embodiment, the expandable member includes a flexible ring. In a related embodiment, the device further includes a handle operably connected to the inner lead member and outer tube at respective proximate ends of the inner lead member and outer tube. The handle has a slider mechanism that is operably connected to the outer tube and includes a slider button configured to move the outer tube in proximal and distal directions to expose and contain the flexible ring within the outer tube, respectively.

In another embodiment, the expandable member includes a T-shaped member having first and second arms, and wherein the T-shaped member extends through the inner lead member. In a related embodiment, the outer tube includes a cavity at a distal end thereof that is continuous with a distal end of the inner lead member. The outer tube includes first and second apertures on opposite sides of its distal end and continuous with the cavity in a related embodiment, wherein the first and second apertures are dimensioned to allow insertion of the first and second arms therethrough, respectively. In a related embodiment, the device further includes a handle operably connected to the inner lead member and outer tube at respective proximate ends of the inner lead member and outer tube. The handle has a slider mechanism that is operably connected to the outer tube and includes a slider button configured to move the outer tube in proximal and distal directions to expose and contain the first and second arms of the T-shaped member within the outer tube, respectively.

According to another exemplary embodiment of the innovation, a device for clearing adhesions in a patient's epidural space includes an inner lead member; an outer tube configured to receive the inner lead member therein; an expandable member operably connected to a distal end of the inner lead member and moveably positioned within the outer tube and a handle operably connected to the inner lead member and outer tube at respective proximate ends of the inner lead member and outer tube. The handle has a slider mechanism that is operably connected to the outer tube and includes a slider button configured to move the outer tube in proximal and distal directions to expose and contain the first and second arms of the expandable member within the outer tube, respectively. The device is moveable between an undeployed position in which the slider button is moved in the distal direction and the expandable member is at least partially contained within the outer tube, and a deployed position in which the slider button is moved in the proximal direction and the expandable member is expanded to extend outside of the outer tube and configured to clear adhesions within the patient's epidural space. In various embodiments, the expandable member includes a flexible ring or a T-shaped member having first and second arms.

According to another exemplary embodiment of the innovation, a device for clearing adhesions in a patient's epidural space includes the following steps (1) providing a device for clearing adhesions in a patient's epidural space, the device having (a) an inner lead member; (b) an outer tube configured to receive the inner lead member therein; and (c) an expandable member operably connected to a distal end of the inner lead member and moveably positioned within the outer tube; (d) the device being moveable between an undeployed position in which the expandable member is at least partially contained within the outer tube, and a deployed position in which the expandable member is expanded to extend outside of the outer tube and configured to clear adhesions within the patient's epidural space; (2) inserting the device in its undeployed position distally into the patient's epidural space; (3) positioning the device in a desired location in the patient's epidural space; (4) changing the device from its undeployed position to its deployed position, wherein the expandable member is expanded; and (5) moving the deployed device in a proximal direction, whereby the expanded expandable member contacts and engages the adhesions and the adhesions are removed from the patient's epidural space. In a related embodiment, the positioning step includes using fluoroscopic control to place the device in the desired location. In another related embodiment, the inserting step includes using a Jamshidi needle to access the patient's epidural space, wherein the device is inserted through the Jamshidi needle.

These and other features and advantages will be apparent from a reading of the following detailed description and a review of the associated drawings. It is also to be understood that both the foregoing general description and the following detailed description are explanatory only and not restrictive of aspects as claimed.

### BRIEF DESCRIPTION OF THE DRAWINGS

The accompanying figures, which are incorporated in and constitute a part of the specification, illustrate preferred embodiments of the invention and, together with the description, serve to explain the objects, advantages, and principles of the invention. Embodiments of the invention are further described but are in no way limited by the following illustrations.

FIG. 1A shows a device according to a first embodiment of the present invention in an undeployed position;

FIG. 1B shows the device of FIG. 1A in a deployed position;

FIG. 1C shows the device of FIG. 1A in its undeployed and deployed positions;

FIG. 2A shows a lumbar section of a patient's spinal cord in which the device of FIG. 1A is used;

FIG. 2B shows the lumbar spinal cord of FIG. 2A with a lumbar interspinous space/epidural space below the spinal cord and a Jamshidi needle for use therein;

FIG. 2C shows the device of FIG. 1A being used within the lumbar interspinous/epidural space in an undeployed position;

FIG. 2D shows the device of FIG. 1A being used within the lumbar interspinous/epidural space in a partially deployed position;

FIG. 2E shows the device of FIG. 1A being used within the lumbar interspinous/epidural space in a partially deployed position and being withdrawn from the lumbar interspinous/epidural space to clear adhesions therein;

FIG. 2F shows the device of FIG. 1A in its undeployed and deployed positions;

FIG. 3A shows a device according to a second embodiment of the present invention in an undeployed position;

FIG. 3B shows the device of FIG. 3A in a deployed position;

FIG. 4 shows a device according to a third embodiment of the present invention;

FIG. 5A shows the device of FIG. 4 in use, in an undeployed position;

FIG. 5B is a detailed view of a distal portion of the device shown in FIG. 5A;

FIG. 5C is a detailed view of a handle and slider mechanism of the device shown in FIG. 5A;

FIG. 5D shows the device of FIG. 4 in use, in a deployed position;

FIG. 6 shows a device according to a fourth embodiment of the present invention;

FIG. 7A shows the device of FIG. 6 in use, in an undeployed position;

FIG. 7B is a detailed view of a distal portion of the device shown in FIG. 7A;

FIG. 7C is a detailed view of a handle and slider mechanism of the device shown in FIG. 7A; and

FIG. 7D shows the device of FIG. 4 in use, in a deployed position.

### DETAILED DESCRIPTION OF THE INVENTION

For clarity and convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

Reference is made in detail to the presently disclosed embodiments of the invention, examples of which are illustrated in accompanying drawings, where in like reference numerals refer to like elements throughout.

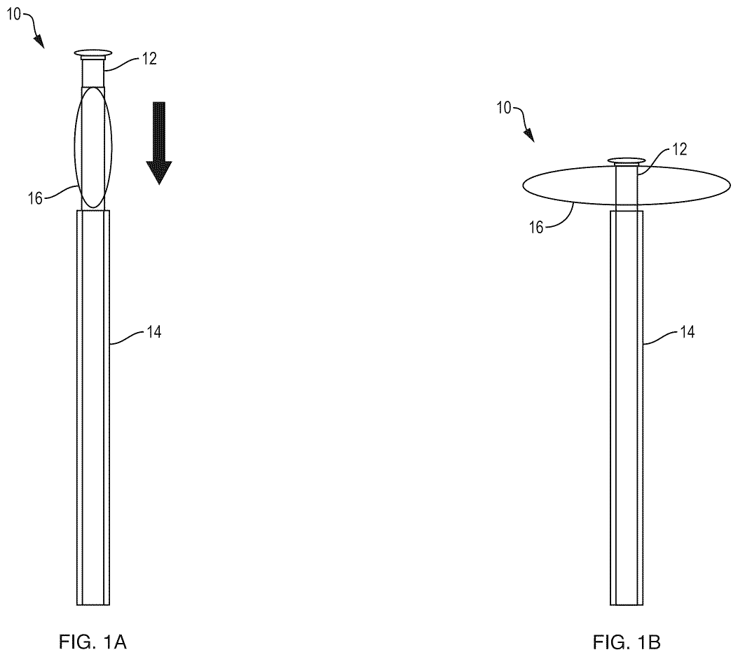

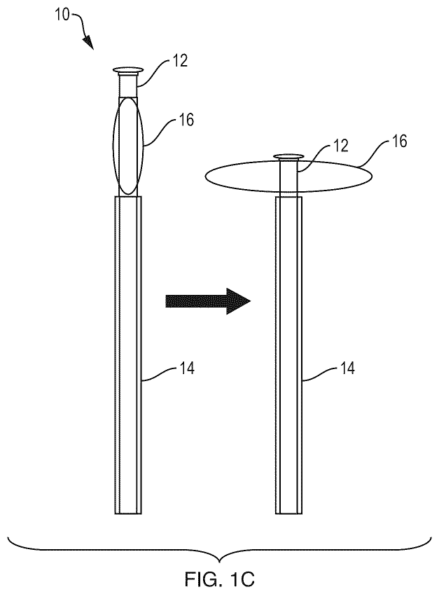

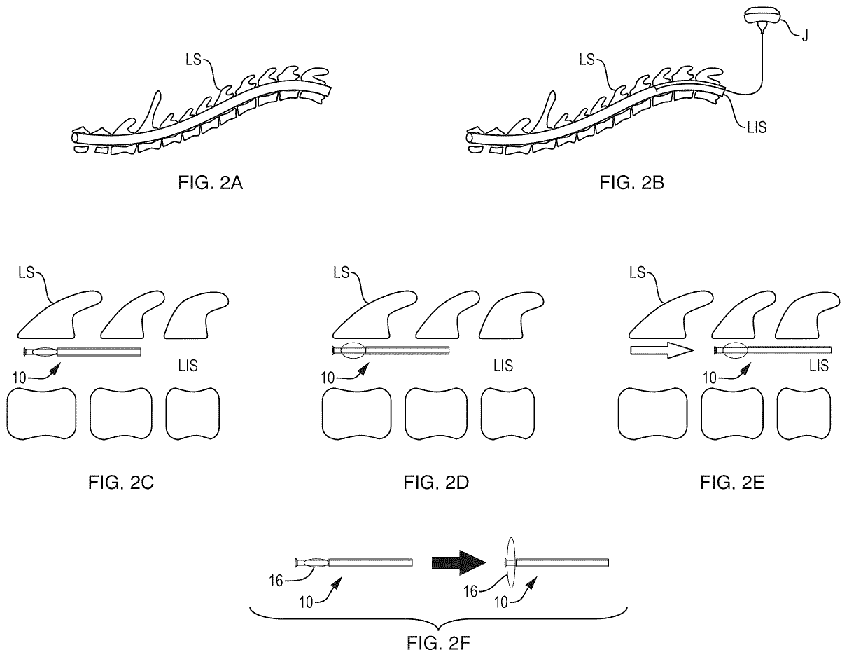

A first embodiment of the invention described herein and illustrated in FIGS. 1A-2F includes a device 10 having a radiopaque, thin, cylindrical inner lead member 12 and a cylindrical outer tube (or cannula) 14 that is configured to receive the lead member 12 therein (i.e., concentrically). The outer tube 14 includes a flexible ring 16 that functions as a pair of outer wings at a distal end thereof. The lead member 12 is also formed as a cylindrical tube that is configured to receive the flexible ring 16 therein. Both the inner lead 12 and outer tube 14 are configured to be deployed together during surgery, as illustrated in FIGS. 2C-2F and further discussed below.

In various embodiments, the lead member 12 and flexible ring 16 are made of a malleable, radiopaque metal with enough elasticity to be deployed and return to its original shape. Non-limiting examples of such materials include steel or an alloy of nickel and titanium (i.e., nitinol). The malleable, radiopaque metal can be either solid or braided.

Usage of the device 10 will now be discussed with reference to FIGS. 2A-2F. After preparing a patient for surgery, a surgeon accesses the portion of patient's brain or spinal cord to be treated with placement/coverage of electrodes. While the figures and description specifically concern a lumbar spine, it will be understood that the devices and methods of the invention may similarly be used with other portions of the patient's nervous system and related anatomy, including, but not limited to, the cervical spine, thoracic spine and brain.

A lumbar section LS of the patient's spinal cord is shown in FIG. 2A. The device 10 is used to free the epidural space is to prepare for the deployment of a shape memory electrode that will cover approximately two vertebral levels (i.e., two adjacent lumbar vertebrae). As further discussed below, the device 10 is advanced to an upper (i.e., more distal) vertebral level, be deployed, moved back to a lower (i.e., more proximal) vertebral level and then be un-deployed for removal from the epidural space.

As shown in FIG. 2B, the surgeon uses a Jamshidi needle J to access the patient's lumbar interspinous space LIS (i.e., epidural space) below the lumbar spinal cord LS to be cleared of adhesions for facilitating the placement of electrodes therein. The surgeon then inserts the device 10 in an undeployed position through the hollow portion of the Jamshidi needle (as shown in FIG. 2B) and distally into the patient's lumbar interspinous space LIS, as shown in FIG. 2C. In an embodiment, the surgeon uses fluoroscopic control to place the device 10 at the desired spinal level (i.e., at or near a designated vertebrae) within the patient's lumbar interspinous space LIS. In an alternate embodiment, the device 10 is used without a Jamshidi needle (i.e., directly inserted into the patient's lumbar interspinous space LIS).

After the surgeon places device 10 in the desired location/spinal level in the patient's lumbar interspinous space LIS (i.e., at the upper/more distal vertebral level), the surgeon begins to deploy the device 10 by pulling the inner lead member 12 away from the outer tube 14 (i.e., in a distal direction) to expose the flexible ring/outer wings 16 of the outer tube 14, as shown in FIG. 2D. The surgeon secures the flexible ring 16 in place in its deployed expanded position for movement.

The surgeon then moves the deployed device 10 in the proximal direction through the patient's lumbar interspinous space LIS (i.e., to the lower/more proximal vertebral level), as shown in FIG. 2E. The flexible ring/outer wings 16 fully extend outward from the outer tube 14 in the deployed position of the device 10, as shown in FIGS. 1B, 1C (right) and 2F (right). By moving the deployed device 10 in the proximal direction, the surgeon clears any adhesions within the lumbar interspinous space LIS. More particularly, extended flexible ring/outer wings 16 of the deployed device 10 contact and engage the adhesions as the deployed device is passed through the lumbar interspinous space LIS in the proximal direction, thereby removing the adhesions with the passage of the device.

After the adhesions have been cleared from the lumbar interspinous space LIS, the surgeon pushes the inner lead member 12 towards from the outer tube 14 (i.e., in a proximal direction) to collapse and retract the flexible ring/outer wings 16 into the outer tube 14. The device 10 is thereby undeployed, and may then be removed from the lumbar interspinous space LIS. After the device 10 has been used to clear adhesions from the lumbar interspinous space LIS as described above, the surgeon may then place the electrodes within the lumbar interspinous space LIS.

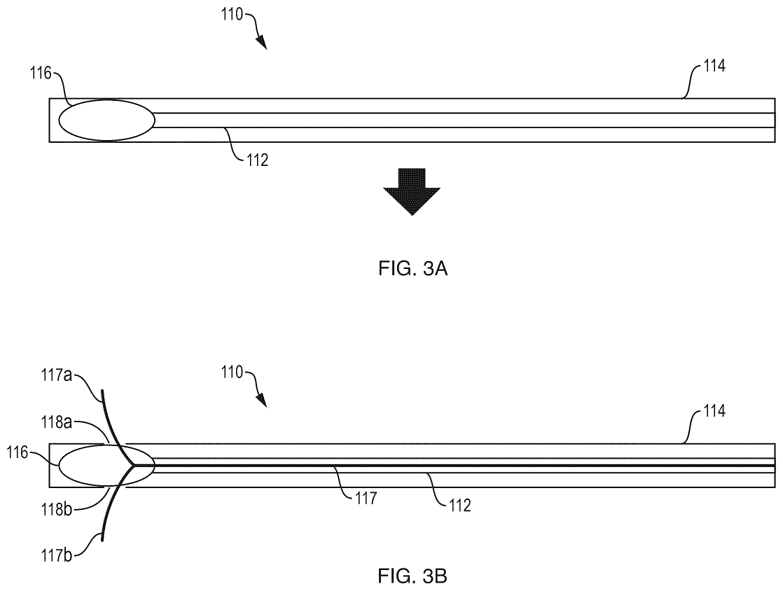

A second embodiment of the invention described herein and illustrated in FIGS. 3A-3B . This embodiment includes a device 110 having a radiopaque, thin, hollow cylindrical inner lead member 112 and a cylindrical outer tube (or cannula) 114 that is configured to receive the lead member 112 therein (i.e., concentrically). A distal end of the outer tube 114 includes a cavity 116 that is continuous with a distal end of the inner lead member 112. The device 110 further includes a T-shaped member 117 that extends through the inner lead member 112 and has two arms, or branches, 117a, 117b at its distal end. The outer tube 114 includes two apertures 118a, 118b on opposite sides of its distal end. The apertures 118a, 118b are continuous with the cavity 116 and dimensioned to allow insertion of the arms 117a, 117b therethrough (see FIG. 3B).

In various embodiments, the lead member 112 and T-shaped member 117 are made of a malleable, radiopaque metal with enough elasticity to be deployed and return to its original shape. Non-limiting examples of such materials include steel or an alloy of nickel and titanium (i.e., nitinol). The malleable, radiopaque metal can be either solid or braided.

In use, a surgeon uses a Jamshidi needle J to access the patient's lumbar interspinous space LIS below the lumbar spinal cord LS as shown in FIGS. 2A and 2B (or other epidural space) to be cleared of adhesions for facilitating the placement of electrodes therein. The surgeon then inserts the device 110 in an undeployed position (as shown in FIG. 3A) through the hollow portion of the Jamshidi needle and distally into the patient's lumbar interspinous space LIS. In an embodiment, the surgeon uses fluoroscopic control to place the device 110 at the desired spinal level within the patient's lumbar interspinous space LIS. In an alternate embodiment, the device 110 is used without a Jamshidi needle (i.e., directly inserted into the patient's lumbar interspinous space LIS).

After the surgeon places the undeployed device 110 in the desired location/spinal level in the patient's lumbar interspinous space LIS (or other epidural space), the surgeon operates the device 110 by moving the T-shaped member 117 distally through the inner lead member 112 and its cavity 116. The surgeon then deploys arms 117a, 117b to extend same through the respective apertures 118a, 118b of the distal end of outer tube 114, as shown in FIG. 3B, whereby the T-shaped member 117 assumes a full T-shape configuration.

The surgeon then moves the deployed device 110 in the proximal direction through the patient's lumbar interspinous space LIS (i.e., from an upper/more distal vertebral level to an adjacent lower/more proximal vertebral level). By moving the deployed device 110 in the proximal direction, the surgeon clears any adhesions within the lumbar interspinous space LIS. More particularly, extended arms 117a, 117b of the T-shaped member 117 of the deployed device 110 contact and engage the adhesions as the deployed device is passed through the lumbar interspinous space LIS in the proximal direction, thereby removing the adhesions with the passage of the device.

After the adhesions have been cleared from the lumbar interspinous space LIS, the surgeon retracts the arms 117a, 117b of the T-shaped member 117 through the respective apertures 118a, 118b of the distal end of the outer tube 114 so that the arms 117a, 117b are contained within the cavity 116 and outer tube 114. The device 110 is thereby undeployed, and may then be removed from the lumbar interspinous space LIS. After the device 110 has been used to clear adhesions from the lumbar interspinous space LIS as described above, the surgeon may then place the electrodes within the lumbar interspinous space LIS.

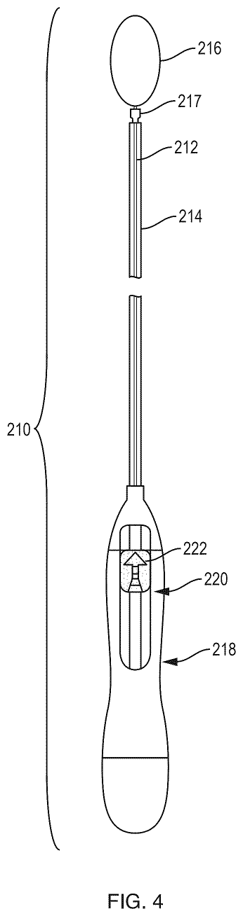

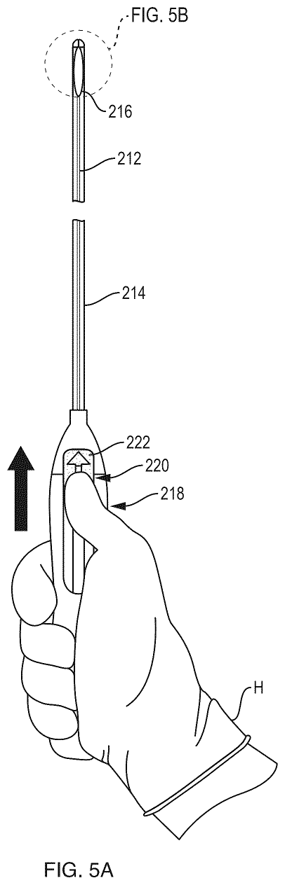

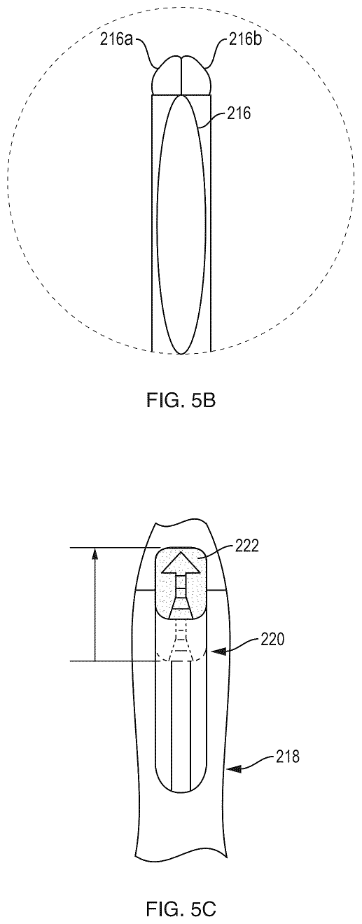

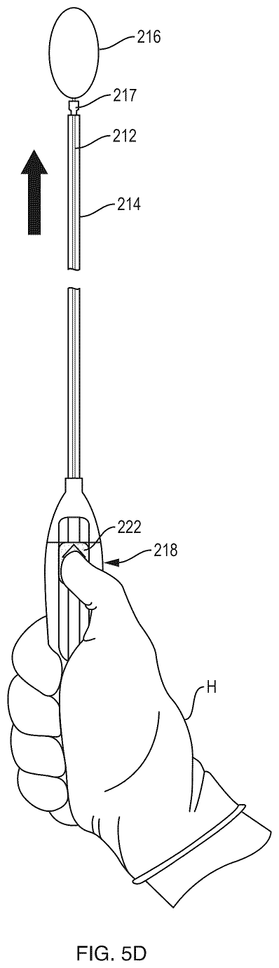

A third embodiment of the invention described herein and illustrated in FIGS. 4-5D . This embodiment includes a device 210 having a radiopaque, thin, hollow cylindrical inner lead member 212 and a cylindrical outer tube (or cannula) 214 that is configured to receive the lead member 212 therein (i.e., concentrically). The device 210 is similar to the device 10 described above and shown in FIGS. 1A-2F in that it includes a includes a flexible ring 216 that functions as a pair of outer wings. The ring 216 has an oval shape as shown. The ring 216 is connected to a distal end of the inner lead member 212 by a connector 217, and the ring 216 and connector 217 are configured to retract into a distal end of the outer tube 214. The ring 216 includes two complimentary collapsible components 216a, 216b (see FIG. 5B) that facilitate the retraction of the ring 216 into the outer tube 214 during operation, as further discussed below.

In various embodiments, the inner lead member 212 and ring 216 are made of a malleable, radiopaque metal with enough elasticity to be deployed and return to its original shape. Non-limiting examples of such materials include steel or an alloy of nickel and titanium (i.e., nitinol). The malleable, radiopaque metal can be either solid or braided.

With continued reference to FIGS. 4-5D , the inner lead member 212 and outer tube 214 are operably connected to a handle 218 at their respective proximate ends. The handle 218 has a slider mechanism 220 that is operably connected to the outer tube 214 and that includes a slider button 222.

In use, a surgeon uses a Jamshidi needle J to access the patient's lumbar interspinous space LIS below the lumbar spinal cord LS as shown in FIGS. 2A and 2B (or other epidural space) to be cleared of adhesions for facilitating the placement of electrodes therein. Using his or her hand H, the surgeon pushes the slider button 222 forward (i.e., in a distal direction) to advance the outer tube 214 in a distal direction and contain/constrain the ring 216, thereby placing the device 210 in its undeployed position. This containment of the ring 216 into the outer tube 214 is facilitated by the collapsible components 216a, 216b moving towards each other (see FIGS. 5A and 5B). The surgeon then inserts the device 210 in this undeployed position through the hollow portion of the Jamshidi needle (as shown in FIG. 2B) and distally into the patient's lumbar interspinous space LIS. In an embodiment, the surgeon uses fluoroscopic control to place the device 210 at the desired spinal level within the patient's lumbar interspinous space LIS. In an alternate embodiment, the device 210 is used without a Jamshidi needle (i.e., directly inserted into the patient's lumbar interspinous space LIS).

After the surgeon places device 210 in the desired location/spinal level in the patient's lumbar interspinous space LIS, the surgeon begins to deploy the device 210 by pushing the slider button 222 backwards (i.e., in a proximal direction), thereby retracting the outer tube 214 to expose the ring 216, as shown in FIGS. 4 and 5D. The surgeon then moves the deployed device 210 in the proximal direction through the patient's lumbar interspinous space LIS (i.e., from an upper/more distal vertebral level to an adjacent lower/more proximal vertebral level). The flexible ring 216 fully extends outward from the outer tube 214 in the deployed position of the device 210, as shown in FIGS. 4 and 5D. By moving the deployed device 210 in the proximal direction, the surgeon clears any adhesions within the lumbar interspinous space LIS. More particularly, extended ring 216 of the deployed device 210 contacts and engages the adhesions as the deployed device is passed through the lumbar interspinous space LIS in the proximal direction, thereby removing the adhesions with the passage of the device.

After the adhesions have been cleared from the lumbar interspinous space LIS, the surgeon again pushes the slider button 222 forward (i.e., in a distal direction) to advance the outer tube 214 in a distal direction and contain/constrain the ring 216, thereby placing the device 210 in its undeployed position. The device 210 may then be removed from the lumbar interspinous space LIS. After the device 210 has been used to clear adhesions from the lumbar interspinous space LIS as described above, the surgeon may then place the electrodes within the lumbar interspinous space LIS.

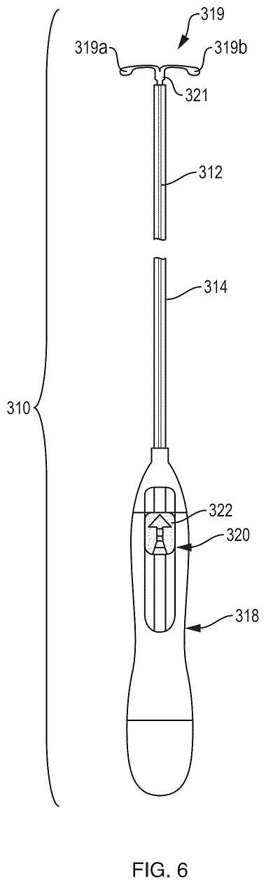

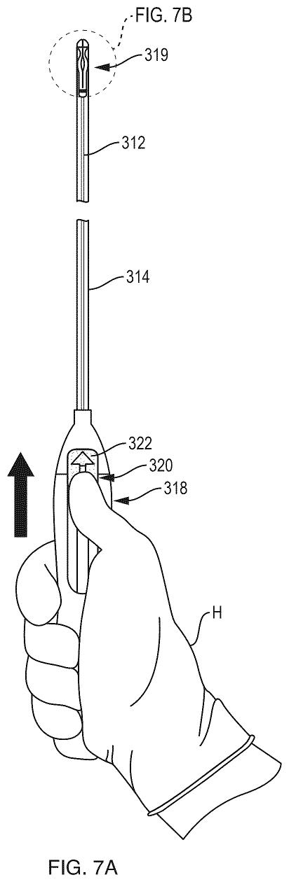

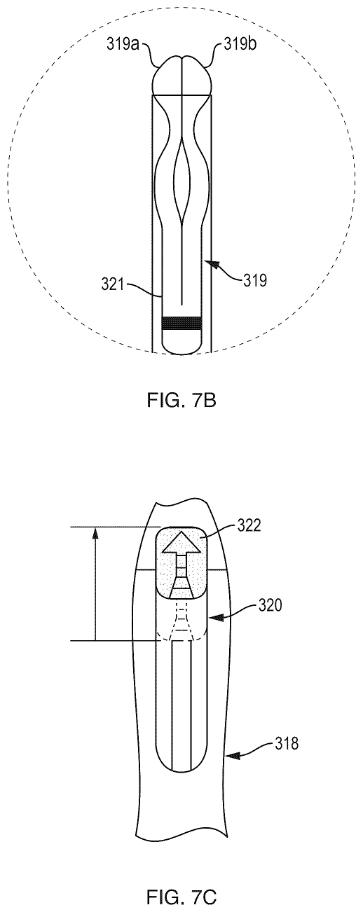

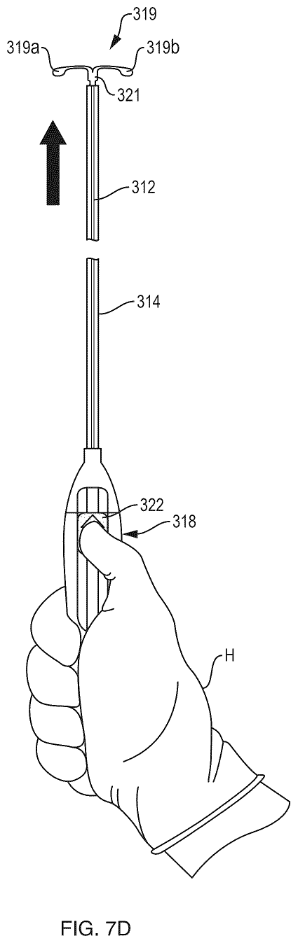

A fourth embodiment of the invention described herein and illustrated in FIGS. 6-7D . This embodiment includes a device 310 having a radiopaque, thin, hollow cylindrical inner lead member 312 and a cylindrical outer tube (or cannula) 314 that is configured to receive the lead member 312 therein (i.e., concentrically). The device 310 is similar to the device 110 described above and shown in FIGS. 3A-3B in that it includes a includes a T-shaped member 319 that has two arms, or arms 319a, 319b. The arms 319a, 319b preferably each have a rounded terminal end, so as to avoid or minimize damage to surrounding tissues.

The T-shaped member 319 is connected to a distal end of the inner lead member 312 by a connector 321, and the T-shaped member 319 (i.e., the arms 319a, 319b and connector 321) is configured to retract into a distal end of the outer tube 314. The two arms 319a, 319b constitute complimentary collapsible components (see FIG. 7B) that facilitate the containment of the T-shaped member 319 within the outer tube 314 during operation, as further discussed below.

In various embodiments, the inner lead member 312 and T-shaped member 319 are made of a malleable, radiopaque metal with enough elasticity to be deployed and return to its original shape. Non-limiting examples of such materials include steel or an alloy of nickel and titanium (i.e., nitinol). The malleable, radiopaque metal can be either solid or braided.

With continued reference to FIGS. 6-7D , the inner lead member 312 and outer tube 314 are operably connected to a handle 318 at their respective proximate ends, similar to the device 210 shown in FIGS. 4-5D . The handle 318 has a slider mechanism 320 that is operably connected to the outer tube 314 and that includes a slider button 322.

In use, a surgeon uses a Jamshidi needle J to access the patient's lumbar interspinous space LIS below the lumbar spinal cord LS as shown in FIGS. 2A and 2B (or other epidural space) to be cleared of adhesions for facilitating the placement of electrodes therein. Using his or her hand H, the surgeon pushes the slider button 322 forward (i.e., in a distal direction) to advance the outer tube 314 in a distal direction and contain/constrain the T-shaped member 319, thereby placing the device 310 in its undeployed position. This containment of the T-shaped member 319 into the outer tube 314 is facilitated by the collapsible arms 319a, 319b moving towards each other (see FIGS. 7A and 7B). The tips of the arms 319a, 319b meet to form a rounded end that extends slightly beyond the outer tube 314 in the illustrated embodiment (see FIG. 7B). The surgeon then inserts the device 310 in this undeployed position through the hollow portion of the Jamshidi needle (as shown in FIG. 2B) and distally into the patient's lumbar interspinous space LIS. In an embodiment, the surgeon uses fluoroscopic control to place the device 310 at the desired spinal level within the patient's lumbar interspinous space LIS. In an alternate embodiment, the device 310 is used without a Jamshidi needle (i.e., directly inserted into the patient's lumbar interspinous space LIS).

After the surgeon places device 310 in the desired location/spinal level in the patient's lumbar interspinous space LIS, the surgeon begins to deploy the device 310 by pushing the slider button 322 backwards (i.e., in a proximal direction), thereby retracting the outer tube 314 to expose the T-shaped member 319, as shown in FIGS. 6 and 7D. The surgeon then moves the deployed device 310 in the proximal direction through the patient's lumbar interspinous space LIS (i.e., from an upper/more distal vertebral level to an adjacent lower/more proximal vertebral level). The arms 319a, 319b fully extend outward from the outer tube 314 in the deployed position of the device 310, as shown in FIGS. 6 and 7D. By moving the deployed device 310 in the proximal direction, the surgeon clears any adhesions within the lumbar interspinous space LIS. More particularly, extended arms 319a, 319b of the deployed device 310 contact and engage the adhesions as the deployed device is passed through the lumbar interspinous space LIS in the proximal direction, thereby removing the adhesions with the passage of the device.

After the adhesions have been cleared from the lumbar interspinous space LIS, the surgeon again pushes the slider button 322 forward (i.e., in a distal direction) to advance the outer tube 314 in a distal direction and contain/constrain the T-shaped member 319 (i.e., by folding the arms 319a, 319b towards each other), thereby placing the device 310 in its undeployed position. The device 310 may then be removed from the lumbar interspinous space LIS. After the device 310 has been used to clear adhesions from the lumbar interspinous space LIS as described above, the surgeon may then place the electrodes within the lumbar interspinous space LIS.

The devices disclosed herein can be used by surgeons or pain management physicians to overcome the challenge of deploying a percutaneous stimulator or recording lead that takes on a larger final conformation once it is in the final epidural location.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Other embodiments of the invention will be apparent to those skilled in the art from consideration of the specification and practice of the invention disclosed herein. Although specific advantages have been enumerated above, various embodiments may include some, none, or all of the enumerated advantages. It is intended that the embodiments described above be considered as exemplary only, with a true scope and spirit of the invention being indicated by the appended claims. Moreover, none of the features disclosed in this specification should be construed as essential elements, and therefore, no disclosed features should be construed as being part of the claimed invention unless the features are specifically recited in the claims. In addition, it should be understood that any of the features disclosed on any particular embodiment may be incorporated in whole or in part on any of the other disclosed embodiments.

In any interpretation of the claims appended hereto, it is noted that no claims or claim elements are intended to invoke or be interpreted under 35 U.S.C. 112(f) unless the words “means for” or “step for” are explicitly used in the particular claim.

In general, any combination of disclosed features, components and methods described herein is possible. Steps of a method can be performed in any order that is physically possible.

All cited references are incorporated by reference herein.

Although embodiments have been disclosed, it is not desired to be limited thereby. Rather, the scope should be determined only by the appended claims.

While various embodiment of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover, though the present disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

In general, any combination of disclosed features, components and methods described herein is possible. Steps of a method can be performed in any order that is physically possible.

I claim:

1. A device for clearing adhesions in a patient's epidural space, the device comprising:
an inner lead member;
an outer tube configured to receive the inner lead member therein; and
an expandable member operably connected to a distal end of the inner lead member and moveably positioned within the outer tube;
the device being moveable between an undeployed position in which the expandable member is at least partially contained within the outer tube, and a deployed position in which the expandable member is expanded to extend outside of the outer tube and configured to clear adhesions within the patient's epidural space.

2. The device of claim 1, wherein the expandable member includes a flexible ring.

3. The device of claim 2, further comprising:
a handle operably connected to the inner lead member and outer tube at respective proximate ends of the inner lead member and outer tube;
wherein the handle has a slider mechanism that is operably connected to the outer tube and includes a slider button configured to move the outer tube in proximal and distal directions to expose and contain the flexible ring within the outer tube, respectively.

4. The device of claim 1, wherein the expandable member includes a T-shaped member having first and second arms, and wherein the T-shaped member extends through the inner lead member.

5. The device of claim 4, wherein the outer tube includes a cavity at a distal end thereof that is continuous with a distal end of the inner lead member.

6. The device of claim 5, wherein the outer tube includes first and second apertures on opposite sides of its distal end and continuous with the cavity, and wherein the first and second apertures are dimensioned to allow insertion of the first and second arms therethrough, respectively.

7. The device of claim 4, further comprising:
a handle operably connected to the inner lead member and outer tube at respective proximate ends of the inner lead member and outer tube;
wherein the handle has a slider mechanism that is operably connected to the outer tube and includes a slider button configured to move the outer tube in proximal and distal directions to expose and contain the first and second arms of the T-shaped member within the outer tube, respectively.

8. The device of claim 1, wherein the lead member and expandable member are made of a malleable, radiopaque metal with enough elasticity to be deployed and return to its original shape.

9. The device of claim 8, wherein the metal is selected from the group consisting of steel and nitinol.

10. The device of claim 8, wherein the metal is selected from the group consisting of a solid configuration and a braided configuration.

11. A device for clearing adhesions in a patient's epidural space, the device comprising:
an inner lead member;
an outer tube configured to receive the inner lead member therein;
an expandable member operably connected to a distal end of the inner lead member and moveably positioned within the outer tube; and
a handle operably connected to the inner lead member and outer tube at respective proximate ends of the inner lead member and outer tube;
wherein the handle has a slider mechanism that is operably connected to the outer tube and includes a slider button configured to move the outer tube in proximal and distal directions to expose and contain the first and second arms of the expandable member within the outer tube, respectively;
the device being moveable between an undeployed position in which the slider button is moved in the distal direction and the expandable member is at least partially contained within the outer tube, and a deployed position in which the slider button is moved in the proximal direction and the expandable member is expanded to extend outside of the outer tube and configured to clear adhesions within the patient's epidural space.

12. The device of claim 11, wherein the expandable member includes a flexible ring.

13. The device of claim 11, wherein the expandable member includes a T-shaped member having first and second arms, and wherein the T-shaped member extends through the inner lead member.

14. The device of claim 11, wherein the lead member and expandable member are made of a malleable, radiopaque metal with enough elasticity to be deployed and return to its original shape.

15. The device of claim 14, wherein the metal is selected from the group consisting of steel and nitinol.

16. The device of claim 14, wherein the metal is selected from the group consisting of a solid configuration and a braided configuration.

17. A method for clearing adhesions in a patient's epidural space, the method comprising the steps of:
providing a device for clearing adhesions in a patient's epidural space, the device including:
an inner lead member;
an outer tube configured to receive the inner lead member therein; and
an expandable member operably connected to a distal end of the inner lead member and moveably positioned within the outer tube;
the device being moveable between an undeployed position in which the expandable member is at least partially contained within the outer tube, and a deployed position in which the expandable member is expanded to extend outside of the outer tube and configured to clear adhesions within the patient's epidural space;
inserting the device in its undeployed position distally into the patient's epidural space;
positioning the device in a desired location in the patient's epidural space;
changing the device from its undeployed position to its deployed position, wherein the expandable member is expanded;
moving the deployed device in a proximal direction, whereby the expanded expandable member contacts and engages the adhesions and the adhesions are removed from the patient's epidural space.

18. The method of claim 17, wherein the positioning step includes using fluoroscopic control to place the device in the desired location.

19. The method of claim 17, wherein the inserting step includes using a Jamshidi needle to access the patient's epidural space, wherein the device is inserted through the Jamshidi needle.
